(19) United States
(12) Patent Application Publication
(10) Pub. No.: US 2024/0366488 A1
(43) Pub. Date: Nov. 7, 2024

(54) COMPOSITIONS AND METHODS FOR PROMOTING HAIR GROWTH

(71) Applicant: LOCUS SOLUTIONS IPCO, LLC, SOLON, OH (US)

(72) Inventors: SEAN FARMER, FT. LAUDERDALE, FL (US); VICTOR A. PERONI, CHARDON, OH (US)

(21) Appl. No.: 18/252,213

(22) PCT Filed: Mar. 31, 2023
(86) PCT No.: PCT/US2023/065198

§ 371 Date: May 9, 2023

Related U.S. Application Data
(60) Provisional application No. 63/325,827, filed on Mar. 31, 2022.

(51) Int. Cl. A61K 8/49; A61K 8/60; A61K 8/99; A61K 31/506; A61K 31/565; A61K 31/58; A61Q 5/02; A61Q 5/12; A61Q 7/00

(52) U.S. Cl. CPC A61K 8/4953; A61K 8/602; A61K 8/99; A61K 31/506; A61K 31/565; A61K 31/58; A61Q 5/02; A61Q 5/12; A61Q 7/00

(57) ABSTRACT

The subject invention provides topical therapeutic compositions and methods of their use for promoting the growth of hair. The subject invention utilizes topical compositions comprising microbial growth by-products and pharmacologic or naturopathic hair growth promoters. In specific embodiments, the microbial growth by-products are biosurfactants.

### CROSS REFERENCE TO RELATED APPLICATION

This application claims priority to U.S. Provisional Patent Application No. 63/325,827, filed Mar. 31, 2022, which is incorporated by reference herein in its entirety.

### BACKGROUND

Human hair is mostly comprised of terminal hairs and vellus hairs. Terminal hairs are coarse, pigmented long hairs in which the bulb, or root, of the hair follicle is seated deep in the dermis. Vellus hairs are fine, thin, non-pigmented short hairs in which the hair bulb is located superficially in the dermis. At the base of the bulb is a papilla, which contains blood vessels and nerves, and provides nourishment to the hair cells to promote growth. The number of papillae is determined at birth and determines the number of hairs that are present.

All hair, both human and animal, passes through a life cycle that includes three phases, namely, the anagen phase, the catagen phase and the telogen phase. The anagen phase is the period of active hair growth. On the scalp, for example, this generally lasts from 3-5 years. The catagen phase is a short transitional phase between the anagen and telogen phases. Again, in the case of scalp hair life cycle, the catagen phase will last approximately 1-2 weeks. The final phase is the telogen phase, where all growth ceases and the hair eventually is shed for preparation of new growth. Scalp hair in the telogen phase lasts around 3-4 months before the hair is shed and a new one begins to grow.

Some individuals experience changes in the hair life cycle and/or hair type, which can lead to hair loss. For example, taking certain medications may cause hair loss by interfering with the normal hair life cycle, such as chemotherapy drugs, contraceptives, steroids, and antibiotics. Some of this hair loss can be reversed with cessation of the medication; however, in some instances hair loss can be a permanent condition.

Skin injuries and the presence of scar tissue can result in permanent hair loss, as well as genetic and hormonal causes, such as alopecia, or “male pattern baldness.” With alopecia, the scalp begins losing hair at the temples and on the crown of the head. This incurable condition is predominantly restricted to males, though it is occasionally observed in women as well.

Through the progression of alopecia, the hairs themselves change from terminal to vellus. Furthermore, with the onset of alopecia, an increasing proportion of the hairs are in the telogen phase with correspondingly fewer in the active growth anagen phase. Alopecia is also associated with the severe diminution of hair follicles, e.g., about a one-third reduction in hair follicles or more. It is thus a combination of the above factors: transition of hairs from terminal to vellus, increased number of telogen hairs-some of which have been shed, and diminution and loss of hair follicles that results in baldness.

One known treatment for alopecia is hair transplantation. Plugs of skin containing hair are transplanted from areas of the scalp where hair is growing to bald areas with reasonable success; however, the procedure is costly in addition to being time-consuming and painful. Other non-drug related treatments include, e.g., ultra-violet radiation, massage, psychiatric treatment, revascularization surgery, acupuncture and exercise therapy. None of these, however, has been generally accepted as being effective.

The most common treatment approach to date for alopecia is drug therapy. For example, the use of a microemulsion cream containing both estradiol and oxandrolone as its active ingredients, and the use of organic silicon, have shown to be useful. In addition, the compounds bimatroprost and minoxidil are useful for stimulation of hair growth and/or the prevention of hair loss.

Bimatroprost is an ophthalmologic medication for treating high eye pressure, glaucoma and for increasing eyelash length. Typically, bimatroprost is applied as a liquid eye drop.

Minoxidil is a treatment for severe hypertension through the dilation of the peripheral vascular system. Dermatologists recognized that prolonged vasodilation of certain areas of the human body other than the scalp sometimes result in increased hair growth, even in the absence of any vasodilating therapeutic agent. For instance, increased hair growth around surgical scars is not uncommon. Similarly, arteriovenous fistula have been known to result in increased vascularity accompanied by enhanced hair growth. First introduced as an oral drug to treat high blood pressure, topical solutions and foam products containing minoxidil were introduced to prevent or treat hair loss.

### BRIEF SUMMARY

The present invention provides topical therapeutic compositions for enhancing skin, scalp and hair health. More specifically, the subject invention provides compositions and methods for promoting hair growth and/or treating hair loss, using microbial growth by-products. Advantageously, the compositions and methods can be useful for subjects experiencing hair loss due to a number of reasons, including alopecia and/or chemotherapy.

In certain embodiments, the present invention provides topical compositions for promoting hair growth, the compositions comprising one or more biological amphiphilic molecules. In certain embodiments, the topical composition comprises the one or more biological amphiphilic molecules in combination with a pharmacologic or naturopathic hair growth promoter.

In a preferred embodiment, the biological amphiphilic molecules are biosurfactants selected from, for example, low molecular weight glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

The one or more biosurfactants can further include any one or a combination of: a modified form, derivative, fraction, isoform, isomer or subtype of a biosurfactant, including forms that are biologically or synthetically modified. In certain embodiments, the biosurfactant is a salt-form biosurfactant.

In certain specific embodiments the biosurfactants are glycolipids, more specifically, sophorolipids (SLP) and/or mannosylerythritol lipids (MEL). In certain embodiments, a combination of SLP and MEL is utilized.

In certain embodiments, the biosurfactants can serve as active ingredients in the topical composition for the purpose of promoting hair growth and/or preventing hair loss. For example, in some embodiments, the biosurfactants can enhance dermal fibroblast proliferation and/or function, and/or can accelerate papillary cell and hair growth.

In certain embodiments, the biosurfactants can serve as adjuvants and/or additives capable of enhancing dermal penetration of active and inactive ingredients in the composition, such as the pharmacologic or naturopathic hair growth promoter, and/or enhancing formulation of a composition, such as through enhanced emulsification.

In certain embodiments, the pharmacologic hair growth promoter of the present compositions is minoxidil or bimatroprost.

In certain exemplary embodiments, the topical composition of the present invention comprises SLP, MEL and minoxidil. Advantageously, a composition having this combination of ingredients possess a surprising synergy for promoting hair growth when compared with each of the individual ingredients on its own.

In some embodiments, the composition comprises live or inactivated microorganisms, growth by-products thereof, and/or culture supernatants of said microorganisms. In certain embodiments, the microorganisms are yeasts, such as, e.g., Starmerella bombicola, Wickerhamomyces anomalus NRRL Y-68030, Meyerozyma guilliermondii, and/or Pseudozyma aphidis. In certain embodiments, the microorganisms are bacteria, such as, e.g., Bacillus coagulans, Bacillus amyloliquefaciens NRRL B-67928 or Bacillus subtilis B4 NRRL B-68031.

In some embodiments, the topical composition can further comprise a dermatologically-acceptable carrier, such as a water-in-oil or oil-in-water emulsion, or an aqueous serum.

In some embodiments, the topical composition can further comprise adjuvants and additives typically found in topical skin and hair care compositions, such as, for example, organic solvents, silicones, stabilizers, thickeners, moisturizers, conditioners, surfactants, colorants, fragrances, pH modifiers, buffers, local anesthetic agents, anti-biofilm agents, antifungal agents, vitamins, minerals, botanicals, extracts, essential oils, retinoids, anti-comedo agents, moisturizers, sunscreens, and/or other therapeutic and non-therapeutic components known to, e.g., heal, replenish, rejuvenate, moisturize, protect and/or improve the skin and scalp, as well as to stimulate and hair growth.

In certain embodiments, the present invention provides methods for promoting hair growth, wherein a topical composition of the present invention is applied directly to an area of a subject's the skin where hair loss has occurred and/or where hair growth is desired. In some embodiments, the topical composition is formulated and applied as a cream, lotion, ointment, gel, paste, essence, tonic, spray, aerosol, solid bar, eye drop or oil, wherein the formulation is suitable for application to the skin of the, e.g., scalp, eyelids, face, limbs or torso.

In some embodiments, the methods can enhance scalp health, prevent hair loss, and/or stimulate hair growth for subjects in need thereof. In some embodiments, the subject is experiencing hair loss as a result of pattern baldness, chemotherapy, a skin injury, a burn, a scar or a medication side-effect.

In certain embodiments, the method can further comprise the application of additional compositions for enhancing hair growth and/or for supplementing the activity of the topical composition as part of a hair growth regimen. For example, in one embodiment, the method can comprise cleansing the scalp or other area of the skin experiencing hair loss with a cleansing composition comprising a mixture of SLP and MEL.

In certain embodiments, the application of the topical composition and/or the cleanser can also be supplemented with administration of an oral compound, such as a DHT (dihydrotestosterone) blocker. In certain embodiments, subjects with pattern baldness experience shrinkage of hair follicles due to binding of the follicles by DHT; thus, in some embodiments, a DHT blocker can help prevent hair loss through this mechanism.

Advantageously, in some embodiments, the use of a multi-part regimen can lead to longer-lasting consistent results for subject experiencing hair loss.

### DETAILED DESCRIPTION

The present invention provides topical therapeutic compositions for enhancing skin, scalp and hair health. More specifically, the subject invention provides compositions and methods for promoting hair growth and/or treating hair loss, using microbial growth by-products. Advantageously, the compositions and methods can be useful for subjects experiencing hair loss due to a number of reasons, including alopecia and/or chemotherapy.

### Selected Definitions

A plant “extract,” as used herein, refers to the material resulting from exposing a plant part to a solvent and removing the solvent, or from using various chemical, immunological, biochemical or physical procedures known to those of skill in the art, including but not limited to, precipitation, steam distillation, centrifugation, filtering, column chromatography, detergent lysis and cold pressing (or expression). Plant extracts can include, for example, essential oils. Plant material can include and part of a plant, including fruits, roots, stems, leaves, flowers, or parts thereof.

As used herein, the term “probiotic” refers to microorganisms, which, when administered in adequate amounts, confer a health benefit on the host. In certain embodiments, probiotics are administered to a subject's digestive tract to confer, for example, digestive benefits to the subject.

The terms “naturopathic,” “natural” and “naturally-derived,” as used in the context of a chemical compound or substance is a material that is found in nature, meaning that it is produced from earth processes or by a living organism. A natural product can also be isolated or purified from its natural source of origin and utilized in, or incorporated into, a variety of applications, including foods, beverages, cosmetics, and supplements. Thus, natural products can be combined with other natural or non-natural products, with which they are not found in nature. A natural product can also be produced in a lab by chemical synthesis, provided no artificial components or ingredients (i.e., synthetic ingredients that cannot be found naturally as a product of the earth or a living organism) are added.

As used herein, the terms “healing” and/or “improving” a condition or disorder refers to eradication, reduction, amelioration or reversal of a sign or symptom of a condition or disorder to any extent or degree, and includes, but does not require, a complete cure of the condition or disorder.

As used herein, “preventing” a condition or disorder refers to avoiding, delaying, forestalling, or minimizing the onset of a particular sign or symptom of the condition or disorder. Prevention can, but is not required to be, absolute or complete, meaning the sign or symptom may still develop at a future time. Prevention can include reducing the severity of the onset of such a condition or disorder, and/or inhibiting the progression of the condition or disorder to a more severe condition or disorder.

According to the subject invention, “promotion” of hair growth means enhancing, or accelerating the rate of hair growth, and/or otherwise creating conditions at a site of hair loss that favor increased hair growth. This can be achieved, in some embodiments, by alteration of the hair life cycle to, for example, increase the anagen or growing stage and/or decrease the telogen phase. Hair growth can include increased length of hair, increased thickness of hair, increased strength of hair and increased numbers of hair. In some embodiments, promoting hair growth can also include preventing and/or slowing hair loss.

As used herein, the term “wound” refers to an injury to tissue caused by, for example, a cut, blow or other impact. According to the subject invention, wounds include injuries to the skin, categorized as “open wounds,” and include, for example, cuts, abrasions, ulcers, lesions, scrapes, crushes, punctures, tears, burns, lacerations, incisions, gunshot wounds, bites, stings and avulsions.

As used herein, the term “burn” refers to a wound caused by thermal (heat or cold), chemical (e.g., from an acid or base), friction, radiation (e.g., sunburn or UV), or electrical sources. A burn may be a “minor” burn, which includes first-degree burns with superficial damage to the outer dermis layer, and second-degree burns, with damage extending down into the epidermal layer of cells. Symptoms of burns include, for example, irritation, blistering, itching, peeling, rashes, redness, and swelling.

As used herein, the term “scar” refers to a mark or growth on the skin where an injury, e.g., a wound, burn, sore, surgical incision or piercing, has not healed properly and fibrous connective tissue has developed in place of normal tissue. Scars can include hypertrophic scars, where an overproduction of collagen creates an area of raised tissue above the surrounding skin; keloids, another form of excessive scarring where the tissue forms into large, protruding neoplasms; atrophic scars, where underlying structural tissue is lost, resulting in a pitted or sunken appearance (e.g., acne scars); and stretch marks, resulting from rapid stretching of the skin during, e.g., pregnancy, growth spurts or skin regeneration.

As used herein, the term “subject” refers to an animal, preferably a mammal. The preferred subject in the context of this invention is a human. The subject can be of any sex and any age or stage of development including infant, toddler, adolescent, teenager, young adult, middle-aged, or senior.

As used herein, “topical” means suitable for local application externally to the skin, or cutaneous application. In some embodiments. “topical” can include ocular application, including to the eyelids.

As used herein, “dermatologically-acceptable,” “cosmetically-acceptable” and “topically-acceptable” are used interchangeably and are intended to mean that a particular component is safe and non-toxic for application to the integument (e.g., skin) at the levels employed. In one embodiment, the components of the composition are recognized as being Generally Regarded as Safe (GRAS).

As used herein, the terms “therapeutically-effective amount,” “effective amount,” and “effective dose” are used to refer to an amount of something (e.g., a compound, a composition, time) that is capable of achieving a desired amount of hair growth in a subject. The actual amount will vary depending on a number of factors including, but not limited to, the particular condition or disorder causing hair loss, the severity of the condition, the size, age, and health of the subject, and the manner of administration.

As used herein, a “microbe-based composition” means a composition that comprises components that were produced as the result of the growth of microorganisms or other cell cultures. Thus, the microbe-based composition may comprise the microbes themselves and/or by-products of microbial growth (e.g., biosurfactants, solvents and/or enzymes). The cells may be in a vegetative state or in spore form, or a mixture of both. The cells may be planktonic or in a biofilm form, or a mixture of both. The cells may be live or inactive, intact or lysed. The cells can be removed from the medium in which they were grown, or present at, for example, a concentration of at least 1×10³, 1×10⁴, 1×10⁵, 1×10⁶, 1×10⁷, 1×10⁸, 1×10⁹, 1×10¹⁰, or 1×10¹¹ or more cells per milliliter of the composition.

In one embodiment, the microbe-based composition may comprise only the medium in which the cells were grown, with the cells removed (although, in some instances, some residual cellular matter may also remain in the medium). The by-products of growth may be present in the medium and can include, for example, metabolites, cell membrane components, expressed proteins, and/or other cellular components. In one embodiment, the microbe-based composition comprises only microbial growth by-products.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers, and other additives and/or adjuvants suitable for a particular application. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

A “metabolite” refers to any substance produced by metabolism (e.g., a growth by-product) or a substance necessary for taking part in a particular metabolic process. Examples of metabolites include, but are not limited to, enzymes, acids, solvents, alcohols, proteins, carbohydrates, vitamins, minerals, microelements, amino acids, polymers, and biosurfactants.

As used herein, the terms “isolated” or “purified,” when used in connection with biological or natural materials such as nucleic acid molecules, polynucleotides, polypeptides, proteins, organic compounds, such as small molecules, microorganism cells/strains, or host cells, means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

As used herein, an “isomer” refers to a molecule with an identical chemical formula to another molecule, but having unique structures. Isomers can be constitutional isomers, where atoms and functional groups are bonded at different locations, and stereoisomers (spatial isomers), where the bond structure is the same but the geometrical positioning of atoms and functional groups in space is different. MEL isomers, for example, can differ in bond type and bond location of the carbohydrate, fatty acid and/or acetyl groups.

As used herein, “surfactant” means a surface-active substance, or a compound that lowers the surface tension (or interfacial tension) between two phases. Surfactants act as, e.g., detergents, wetting agents, emulsifiers, foaming agents, and/or dispersants. By “biosurfactant” is meant a surface active agent produced by a living organism, and/or produced using naturally-derived substrates.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein. Other features and advantages of the invention will be apparent from the following description of the preferred embodiments thereof, and from the claims. All references cited herein are hereby incorporated by reference.

### Topical Hair Growth Compositions

In certain embodiments, the present invention provides topical compositions for promoting hair growth, the compositions comprising one or more biological amphiphilic molecules in combination with a pharmacologic or naturopathic hair growth promoter.

In a preferred embodiment, the biological amphiphilic molecules are biosurfactants selected from, for example, low molecular weight glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

Biosurfactants are amphiphiles, meaning they comprise both a polar (hydrophilic) moiety and non-polar (hydrophobic) group. Due to their amphiphilic structure, biosurfactants reduce the surface and interfacial tensions between the molecules of liquids, solids, and gases.

Additionally, biosurfactants accumulate at interfaces, thus leading to the formation of aggregated micellar structures in solution. The ability of biosurfactants to form pores and destabilize biological membranes permits their use as, e.g., antibacterial and antifungal agents, as well as delivery agents for other compounds across, e.g., membranes. Furthermore, biosurfactants are biodegradable, have low toxicity, and can be produced using low-cost renewable resources. They can inhibit microbial adhesion to a variety of surfaces, prevent the formation of biofilms, and can have powerful emulsifying and demulsifying properties.

The one or more biosurfactants can further include any one or a combination of: a modified form, derivative, fraction, isoform, isomer or subtype of a biosurfactant, including forms that are biologically or synthetically modified. In one embodiment, the one or more biosurfactants are present in the composition in critical micelle concentration (CMC).

In some embodiments, the amphiphilic molecules are utilized in a crude form, wherein the molecule is present in the growth medium (e.g., broth) in which a amphiphile-producing microorganism is cultivated and is collected therefrom without purification. The crude form can comprise, for example, at least 0.001%, 1%, 5%, 10%, 20%, 30%, 40%, 50%, 60%, 70%, 80%, 90% or 99% amphiphilic molecules in the growth medium. In alternate embodiments, the amphiphilic molecules are purified from the products of cultivation.

In certain embodiments, the biosurfactants can serve as active ingredients in the topical composition for the purpose of promoting hair growth and/or preventing hair loss. For example, in some embodiments, the biosurfactants can enhance dermal fibroblast proliferation and/or function, and/or can accelerate papillary cell and hair growth. In some embodiments, the biosurfactants according to the present invention are also capable of one or more of the following: killing pathogenic agents in/on the skin/scalp, modulating the skin's immune system, killing melanocytes to allow for replacement cells to grow, and reducing oxidative stress.

In certain embodiments, the biosurfactants can serve as adjuvants and/or additives capable of enhancing dermal penetration of active and inactive ingredients in the composition, such as the pharmacologic or naturopathic hair growth promoter, and/or enhancing formulation of a composition, such as through enhanced emulsification.

In preferred embodiments, the biosurfactants are sophorolipids (SLP) and/or mannosylerythritol lipids (MEL), which are both glycolipid biosurfactants produced by certain yeasts. Glycolipids generally comprises a mono- or oligosaccharide group attached to a sphingolipid or a glycerol group that can be acetylated or alkylated, and one or more fatty acids.

SLP are produced by, for example, Starmerella clade yeasts, including Candida apicola and Starmerella bombicola. In some embodiments, Wickerhamomyces anomalus is a SLP-producer (e.g., W. anomalus NRRL Y-68030). SLP consist of a disaccharide sophorose linked to long chain hydroxy fatty acids. These SLPs are a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. The fatty acid carboxyl group can be free (acidic or open form) or internally esterified at the 4″-position (lactone form).

SLP have environmental compatibility, high biodegradability, low toxicity, high selectivity and specific activity in a broad range of temperature, pH and salinity conditions. Additionally, in some embodiments, SLP can be advantageous for applications in cosmetics and dermatology due to their small micelle size, which can help facilitate the movement of the micelle, and compounds enclosed therein, through nanoscale pores and spaces (e.g., between epithelial cells, and into biofilm matrices). In certain embodiments, the micelle size of a SLP is less than 100 nm, less than 50 nm, less than 20 nm, less than 15 nm, less than 10 nm, or less than 5 nm.

In certain embodiments, the SLP according to the subject invention are represented by General Formula (1) and/or General Formula (2), and are obtained as a collection of 30 or more types of structural homologues having different fatty acid chain lengths (R³), and, in some instances, having an acetylation or protonation at R¹ and/or R².

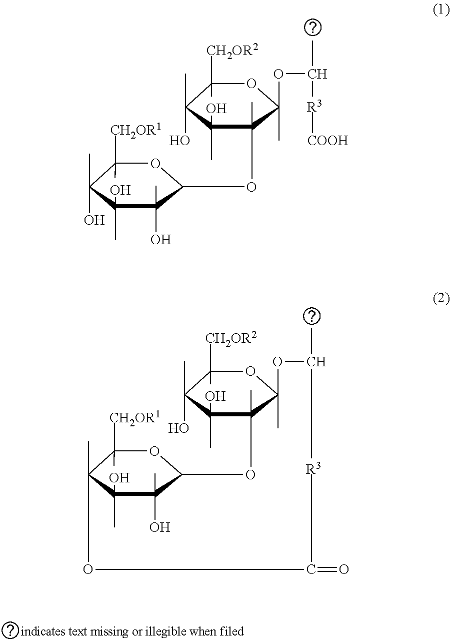

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond and may have one or more Substituents.

Non-limiting examples of the Substituents include halogen atoms, hydroxyl, lower (C1-6)alkyl groups, halo lower (C1-6)alkyl groups, hydroxy lower (C1-6)alkyl groups, halo lower (C1-6)alkoxy groups, and the like. R³ typically has 11 to 20 carbon atoms, preferably 13 to 17 carbon atoms, and more preferably 14 to 16 carbon atoms.

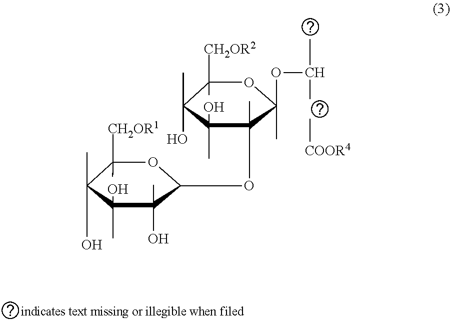

In certain embodiments, the SLP according to the subject invention are salt-form SLP represented by General Formula (3), where R⁴=a metal ion, e.g., Na, K, Mg, Cu or Ca.

Advantageously, SLP can have several benefits as active or inactive ingredients in topical compositions. These can include, for example, emulsifying oil-in-water or water-in-oil mixtures; reducing inflammation and oxidative stress; removal of damage keratinocytes from upper layers of skin; enhancing wound healing through antimicrobial and anti-inflammatory effects; promoting the metabolism of fibroblasts; improving collagen synthesis and toning/restructuring the skin; stimulating leptin synthesis through adipocytes and helping reduce subcutaneous fat overload causing cellulite; inhibiting elastase activity and reducing the appearance of wrinkles; desquamating and depigmenting spots on the skin and inhibiting melanogenisis; controlling microbial dandruff, acne and body odor; and/or reducing inflammatory conditions, such as dermatitis, eczema, and psoriasis.

In certain preferred embodiments, the SIP comprises a mixture of lactonic and linear SLP, e.g., a mixture of SLP according to General Formulas (1) and (2). In some embodiments, the ratio of lactonic to linear SIP is between 50:50 to 10:90, between 40:60 and 20:80, or about 30:70 lactonic to linear.

In one embodiment, the glycolipid is a MEL. MEL comprise either 4-0)-B-D-mannopyranosyl-meso-erythritol or 1-O-B-D-mannopyranosyl-meso-erythritol as the hydrophilic moiety, and fatty acid groups and/or acetyl groups as the hydrophobic moiety. One or two of the hydroxyls, typically at the C4 and/or C6 of the mannose residue, can be acetylated. Furthermore, there can be one to three esterified fatty acids, from 8 to 12 carbons or more in chain length.

MEL and MEL-like substances (e.g., mannose-based substances) are produced mainly by Pseudozyma spp. and Ustilago spp., with significant variability among MEI, structures produced by each species. Certain mannose-based substances having similar properties to MEL can also be produced by Meyerozyma guilliermondii yeasts.

MEL are non-toxic and are stable at wide temperatures and pH ranges. Furthermore, MEL can be used without any additional preservatives

MEL can be produced in more than 93 different combinations that fall under 5 main categories: MEL A, MEL B, MEL D, Tri-acetylated MEL A, and Tri-acetylated MEL B/C. These molecules can be modified, either synthetically or in nature. For example, MEL can comprise different carbon-length chains or different numbers of acetyl and/or fatty acid groups.

MEL molecules and/or modified forms thereof according to the subject invention can include, for example, tri-acylated, di-acylated, mono-acylated, tri-acetylated, di-acetylated, mono-acetylated and non-acetylated MEL, as well as stereoisomers and/or constitutional isomers thereof.

Other mannose-based substances/MEL-like substances that exhibit similar structures and similar properties, can also be used according to the subject invention, e.g., mannosyl-mannitol lipids (MML), mannosyl-arabitol lipids (MAL), and/or mannosyl-ribitol lipids (MRL).

Advantageously, MEL can have several benefits as active or inactive ingredients in topical compositions. These can include, for example, reducing inflammation of the skin; preventing cell damage caused by the use of synthetic surfactants, such as SDS; stimulating hair bulb cells and hair growth; repairing and/or strengthening damaged hair (particularly, e.g., MEL-A and MEL-B); increasing the viability of fibroblast cells and papilla cells (particularly, e.g., MEL-A); reducing perspiration and assisting in skin moisture barrier functioning; and/or decreasing melanin content in age spots

In a specific preferred embodiment, the topical composition of the subject invention comprises a mixture of SLP and MEL.

In preferred embodiments, the concentration of SLP with respect to the total topical composition is about 100 to 500 ppm, about 125 to 375 ppm, about 150 to 300 ppm, about 180 to 250 ppm, or about 200 ppm.

In preferred embodiments, the concentration of MEL (or MEL-like substances) with respect to the total topical composition is from 0.01 to 100 ppm, about 1 to 75 ppm, about 2 to 50 ppm, about 3 to 25 ppm, about 5 to 10 ppm, about 4 to 6 ppm, about 5 ppm or 5 ppm or less.

In one embodiment, the biosurfactants can comprise one or more lipopeptides, such as, for example, surfactin, iturin, fengycin, arthrofactin, viscosin, amphisin, syringomycin, and/or lichenysin.

In a specific embodiment, the lipopeptide biosurfactant is surfactin. Lipopeptides are produced by a variety of probiotics and non-pathogenic bacteria, such as, e.g., Bacillus natto, Bacillus coagulans, Bacillus subtilis, Bacillus amyloliquefaciens, lactic acid bacteria, and others. In some embodiments, the lipopeptide is produced by a specific strain of Bacillus, e.g., B. amyloliquefaciens NRRL B-67928 or B. subtilis B4 NRRL B-68031.

In certain embodiments, the composition according to the present invention further comprises one or more pharmacologic or naturopathic hair growth promoters, including, for example, minoxidil, bimatroprost, finasteride, dihydrotestosterone, biotin, fibroblast growth factors, keratinocyte growth factors, basic fibroblast growth factors, platelet-derived growth factors, spironolactone, vitamins and minerals (e.g., B vitamins, vitamin E, vitamin A, vitamin C, vitamin D, iron, folic acid, zinc), retinol, collagen peptides, omega-3, -6 and -9 fatty acids, plant extracts (e.g., from Citrus limon L., Fragaria ananassa L., Secale cereale L., Epimedium spp., Geranium sibiricum L., Olea europaea L., Camellia sinensis, Coffea arabica, Carthamus tinctorius L., Panax ginseng Mayer, Hottuynia cordata Thunb., Sophora glavescens Aiton, Illicium anisatum L., Illicium verum Hook. f., Hordeum vulgare L., Nelumbinis semen, Chamaecyparis obtuse, Polygonum multiflorum, Alnus sibirica, Malva verticillate, Magnolia officinalis, Angelica gigas, Caesalpinia sappan, Broussonetia papyrifera, Thuja orientalis, Ipomoea batatas, Ishige sinicola, Prunis mira Kochne, Saussurea lappa, Cornus officinalis, Anemarrhena asphodeloides, Salvia plebeian, Undariopsis peterseniana, Pueraria thomsonii, Platycladus orientalis, Polygonum multiforum, Rosmarinus officinalis, Avicennia marina, Ecklonia cava, Sabal serrulatum, etc.), and bioactive compounds (e.g., sinapic acid, icariin, corilagin, gallic acid, oleuropein, caffeine, hydroxysafflor yellow A, linoleic acid, B-sitosterol, quercitrin, L-maackiain, medicarpin, shikimic acid, procyanidin (including B2 and B3) ginsenoside Rb1, anthraquinone, flavonoids, tannins, saponins, a-terpinyl acetate, sabinene, isobornyl acetate, limonene, β-D-glucoside, emodin, oregonin, myristoleic acid, liposomal honokiol, decursin, 3-deoxysappanchalcone, 7-hydroxycoumarin, protocatechuate acid, ferulic acid, protocatechuic acid, epicatechin, β-sitosterol, kaempferol, isoquercetin, octaphlorethol A, vitamin E, olcic acid, costunolide, morroniside, timosaponin BII, phenolic acids, terpenes, terpenoids, saponins, myricitrin, isoquercitrin, quercitrin, myricetin, afzelin, quercetin, kaempferol, amentoflavone, hinokiflavone, ginsenoside Ro, rhyscion, avicequinone C, dieckol, etc.).

In certain embodiments, the pharmacologic or naturopathic hair growth promoter is a compound that can stimulate resting follicles (telogen phase) to growing follicles (anagen phase) (e.g., extension of the anagen phase).

In some embodiments, the pharmacologic or naturopathic hair growth promoter is a compound that can promote expression of growth factors, reduction in inflammatory cytokines, activation of Wnt/β-catenin signaling, promotion of hedgehog pathway signaling (Shh), inhibition of 5α-reductase, and/or improvement in cell cycle progression.

In a specific embodiment, the pharmacologic hair growth promoter is minoxidil.

In certain embodiments, the pharmacologic or naturopathic hair growth promoter is present in the topical composition at a concentration from 0.001% to 90% of the total composition by weight, from 0.01% to 50%, from 0.05% to 10%, or about 5%.

In certain exemplary embodiments, the topical composition of the present invention comprises SLP, MEL, and minoxidil. Advantageously, a composition having this combination of ingredients possess a surprising synergy for promoting hair growth when compared with each of the individual ingredients on its own.

In certain embodiments, in addition to having therapeutic benefits themselves, the biosurfactants can help increase the transdermal absorption of minoxidil to enhance the benefits, such as, e.g., alteration of hair growth phases and hair thickness. In certain embodiments, the biosurfactants can form a micelle, liposome or vesicle around the minoxidil to enhance its absorption through the skin. In certain embodiments, the biosurfactants can bind with the minoxidil to facilitate its absorption through the skin.

In a specific example, the composition comprises 200 ppm SLP, 5 ppm MEL, and 5% minoxidil, wherein the SLP comprises a ratio of linear to lactonic SLP of 70:30.

In some embodiments, the topical composition of the present invention comprises live or inactivated microorganisms capable of producing growth by-products useful for hair growth, skin healing and rejuvenation, and/or supernatants derived from cultivation of such a microorganism. In certain embodiments, the microorganisms are yeasts, such as, e.g., Starmerella bombicola, Wickerhamomyces anomalus (e.g., NRRL Y-68030), Meyerozyma (Pichia) guilliermondii, and/or Pseudozyma aphidis. In one specific embodiment, the composition comprises live or inactivated Wickerhamomyces anomalus.

In certain embodiments, the microorganisms are bacteria, e.g., Lactobacillus spp., Bifido spp., Lactococcus spp., Streptococcus spp., and Bacillus spp., e.g., Bacillus acidiceler, B. acidicola, B. acidiproducens, B. acidocaldarius, B. acidoterrestrisr, B. aeolius, B. aerius, B. aerophilus, B. agaradhaerens, B. agri, B. aidingensis, B. akibai, B. alcalophilus, B. algicola, B. alginolyticus, B. alkalidiazotrophicus, B. alkalinitrilicus, B. alkalisediminis, B. alkalitelluris, B. altitudinis, B. alveayuensis, B. alvei, B. amyloliquefaciens, B. a. subsp. amyloliquefaciens, B. a. subsp. plantarum, B. mylolyticus, B. andreesenii, B. aneurinilyticus, B. anthracia, B. aquimaris, B. arenosi, B. arseniciselenatis, B. arsenicus, B. aurantiacus, B. arvi, B. aryabhattai, B. asahii, B. atrophaeus, B. axarquiensis, B. azotofixans. B. azotoformans, B. badius, B. barbaricus, B. bataviensis, B. beijingensis, B. benzoevorans, B. beringensis, B. berkeleyi, B. beveridgei, B. bogoriensis, B. boroniphilus, B. borstelensis, B. brevis Migula, B. butanolivorans. B. canaveralius, B. carboniphilus, B. cecembensis, B. cellulosilyticus, B. centrosporus, B. cereus, B. chagannorensis, B. chitinolyticus, B. chondroitinus, B. choshinensis, B. chungangensis, B. cibi, B. circulans, B. clarkii, B. clausii, B. coagulans, B. coahuilensis, B. cohnii, B. composti. B. curdlanolyticus, B. cycloheptanicus, B. cytotoxicus, B. daliensis, B. decisifrondis, B. decolorationis, B. deserti, B. dipsosauri, B. drentensis, B. edaphicus, B. ehimensis, B. eiseniae, B. enclensis, B. endophyticus, B. endoradicis. B. farraginis, B. fastidiosus, B. fengqiuensis, B. firmus, B. flexus. B. foraminis, B. fordii, B. formosus, B. fortis, B. fumarioli, B. funiculus, B. fusiformis, B. galactophilus, B. galactosidilyticus, B. galliciensis, B. gelatini, B. gibsonii, B. ginsengi. B. ginsengihumi, B. ginsengisoli, B. globisporus, B. g. subsp. globisporus, B. g. subsp. marinus, B. glucanolyticus. B. gordonae. B. gottheilii, B. graminis, B. halmapalus, B. haloalkaliphilus, B. halochares, B. halodenitrificans, B. halodurans, B. halophilus, B. halosaccharovorans, B. hemicellulosilyticus, B. hemicentroti, B. herbersteinensis, B. horikoshii, B. horneckiae, B. horti, B. huizhouensis, B. humi, B. hwajinpoensis, B. idriensis, B. indicus, B. infantis, B. infernus. B. insolitus, B. invictae, B. iranensis, B. isabeliae, B. isronensis, B. jeotgali, B. kaustophilus, B. kobensis, B. kochii, B. kokeshiiformis, B. koreensis, B. korlensis, B. kribbensis, B. krulwichiae, B. laevolacticus, B. larvae, B. laterosporus, B. lautus, B. lehensis, B. lentimorbus, B. lentus, B. licheniformis, B. ligniniphilus, B. litoralis, B. locisalis. B. luciferensis, B. luteolus, B. luteus, B. macauensis, B. macerans, B. macquariensis, B. macyae, B. malacitensis, B. mannanilyticus, B. marisflavi, B. marismortui, B. marmarensis, B. massiliensis, B. megaterium. B. mesonae, B. methanolicus, B. methylotrophicus, B. migulanus, B. mojavensis, B. mucilaginosus, B. muralis, B. murimartini, B. mycoides, B. naganoensis, B. nanhaiensis, B. nanhaiisediminis, B. nealsonii, B. neidei, B. neizhouensis, B. niabensis, B. niacini, B. novalis, B. oceanisediminis, B. odysseyi, B. okhensis, B. okuhidensis, B. oleronius, B. oryzaecorticis, B. oshimensis, B. pabuli, B. pakistanensis, B. pallidus, B. pallidus, B. panacisoli, B. panaciterrae, B. pantothenticus, B. parabrevis. B. paraflexus, B. pasteurii, B. patagoniensis, B. peoriae, B. persepolensis, B. persicus, B. pervagus, B. plakortidis, B. pocheonensis, B. polygoni. B. polymyxa, B. popilliae, B. pseudalcalophilus, B. pseudofirmus, B. pseudomycoides, B. psychrodurans, B. psychrophilus, B. psychrosaccharolyticus, B. psychrotolerans, B. pulvifaciens, B. pumilus, B. purgationiresistens, B. pycnus, B. qingdaonensis, B. qingshengii, B. reuszeri, B. rhizosphaerae, B. rigui, B. ruris, B. safensis, B. salarius, B. salexigens, B. saliphilus, B. schlegelii, B. sediminis, B. selenatarsenatis, B. selenitireducens, B. seohaeanensis, B. shacheensis, B. shackletonii, B. siamensis, B. silvestris. B. simplex, B. siralis, B. smithii, B. soli, B. solimangrovi, B. solisalsi, B. songklensis, B. sonorensis, B. sphaericus, B. sporothermodurans. B. stearothermophilus, B. stratosphericus, B. subterraneus, B. subtilis, B. s. subsp. inaquosorum, B. s. subsp. spizizenii, B. s. subsp. subtilis, B. taeanensis, B. tequilensis, B. thermantarcticus, B. thermoaerophilus, B. thermoamylovorans, B. thermocatenulatus, B. thermocloacae, B. thermocopriae, B. thermodenitrificans, B. thermoglucosidasius, B. thermolactis, B. thermoleovorans, B. thermophilus, B. thermoruber, B. thermosphaericus, B. thiaminolyticus, B. thioparans, B. thuringiensis, B. tianshenii. B. trypoxylicola, B. tusciae, B. validus, B. vallismortis, B. vedderi, B. velezensis, B. vietnamensis, B. vireti, B. vulcani, B. wakoensis. B. weihenstephanensis, B. xiamenensis, B. xiaoxiensis, B. zhanjiangensis, B. amyloliquefaciens NRRL B-67928, B. subtilis B4 NRRL B-68031, and/or Bacillus coagulans (e.g., BC-30).

In some embodiments, the composition comprises a microbial culture supernatant in place of the pharmacologic or naturopathic hair growth promoter. For example, in one specific embodiment, the composition comprises SLP, MEL, and a supernatant of a Bacillus sp., such as Bacillus coagulans.

Additional microbial growth by-products useful according to the present invention include mannoprotein, beta-glucan, enzymes, and other metabolites that have bio-emulsifying and surface/interfacial tension-reducing properties.

In some embodiments, the topical cosmetic composition can comprise therapeutically effective amounts of enzymes and/or proteins produced by microorganisms. For example, from about 0.001% to about 20% by weight, about 0.01% to about 15% by weight, or about 0.05% to about 10% by weight, of one or more enzymes and/or proteins can be included. These can include, but are not limited to, exo-beta-1,3-glucanase; chitinase; esterases; lipases; glycosidases; amylases; and proteases beneficial for improving skin health.

In some embodiments, the topical therapeutic composition can further comprise a dermatologically-acceptable carrier or vehicle.

The carrier or vehicle may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, preferably organomodified derivatives including PDMS, dimethicone copolyol, dimethiconols, and amodimethiconols; hydrocarbon oils such as mineral oil, petrolatum, isocicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 1% to about 99% by weight of the composition, from 10% to about 85%, from 25% to 75%, or from 50% to about 65%.

As used herein, the term “oil” includes silicone oils unless otherwise indicated. The emulsion may include an emulsifier, such as a nonionic, anionic or amphoteric surfactant, or a gallant, typically in an amount from about 0.001% to about 5% by weight.

In some embodiments, the topical composition can further comprise additional adjuvants and additives commonly included in skin and hair care compositions, such as, for example, organic solvents, stabilizers, silicones, thickeners, softeners, sunscreens, moisturizers, fragrances or others described herein. The amounts of each ingredient, whether active or inactive, are those conventionally used in the cosmetic field to achieve their intended purpose, and typically range from about 0.0001% to about 25%, or from about 0.001% to about 20% of the composition, although the amounts may fall outside of these ranges. The nature of these ingredients and their amounts must be compatible with the production and function of the compositions of the disclosure.

In one embodiment, the composition may include additional skin actives, including but not limited to, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors, to name only a few.

In one embodiment, the composition may include anti-aging components, including, but not limited to, botanicals (e.g., Butea frondosa extract); phytol; phytonic acid; phospholipids; silicones; petrolatum; triglycerides; omega fatty acids; retinoids: hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof.)

In one embodiment, the composition may include an exfoliating agent. Suitable exfoliating agents include, for example, alpha-hydroxy acids, beta-hydroxy acids, oxa-acids, oxadiacids, and their derivatives, such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

In one embodiment, the composition may comprise one or more antioxidants. Suitable antioxidants include, for example, compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Antioxidants may comprise, individually or collectively, from about 0.001% to about 10% (w/w), or from about 0.01% to about 5% (w/w) of the total weight of the composition.

Non-biological surfactants can also be added to the formulation. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropul betaine (CAPB)), sultaines, sacrosinates, isethionates, taurates, ethoxylated sorbitan esters, alkanolamides and amino-acid based surfactants.

Viscosity modifiers can also be added to the compositions, including, for example, cocamide DEA, oleamide DEA, sodium chloride, cellulosic polymers, polyacrylates, ethoxylated esters, alcohol, glycols, xylene sulfonates, polysorbate 20, alkanolamides, and cellulose derivatives (e.g., hydroxypropyl methylcellulose and hydroxyethyl cellulose).

Polymers can also be added, including, for example, xanthan gum, guar gum, polyquaternium-10, PEG-120 methyl glucose dioleate, PEG-150 distearate, PEG-150 polyglyceryl-2 tristearate and PEG-150 pentaerythrityl tetrastearate

A sunscreen or combination of sunscreens may be included to protect the skin and scalp from both UVA and UVB rays. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

The composition can include pH adjusters (e.g., citric acid, ethanolamine, sodium hydroxide, etc.) to be formulated within a wide range of pH levels. In one embodiment, the pH of the topical composition ranges from 1.0 to 13.0. In some embodiments, the pH of the topical composition ranges from 2.0 to 12.0. Other pH ranges suitable for the subject composition include from 3.5 to 7.0, or from 7.0 to 10.5. Suitable pH adjusters such as sodium hydroxide, citric acid and triethanolamine may be added to bring the pH within the desired range.

The composition may optionally comprise other components, additives or adjuvants known to those skilled in the art including, but not limited to: skin penetration enhancers; emollients (e.g., isopropyl myristate, petrolatum, volatile or non-volatile silicones oils, such as methicone and dimethicone, ester oils, mineral oils, and fatty acid esters); humectants (e.g., glycerin, hexylene glycol, caprylyl glycol); skin plumpers (e.g., palmitoyl oligopeptide, collagen, collagen and/or glycosaminoglycan (GAG) enhancing agents); anti-inflammatory agents (e.g., Aloe vera, bioflavonoids, diclofenac, salicylic acid); chelating agents (e.g., EDTA or a salt thereof, such as disodium EDTA); vitamins (e.g., tocopherol and ascorbic acid); vitamin derivatives (e.g., ascorbyl monopalmitate, tocopheryl acetate, Vitamin E palmitate); thickeners (e.g., hydroxyalkyl cellulose, carboxymethylcellulose, carbombers, and vegetable gums, such as xanthan gum); gelling agents (e.g., ester-terminated polyester amides); structuring agents; proteins; immune modulators (e.g., corticosteroids and non-steroidal immune modulators).

Other components that may be included are film formers, moisturizers, minerals, viscosity and/or rheology modifiers, insect repellents, skin cooling compounds, skin protectants, lubricants, preservatives, pearls, chromalites, micas, conditioners, anti-allergenics, antimicrobials (e.g., antifungals, antivirals, antibacterials), antiseptics, pharmaceutical agents, photostabilizing agents, surface smoothers, optical diffusers, and exfoliation promoters. Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute from about 0.01% to about 20% of the total weight of the composition.

The composition may be formulated as a suspension, emulsion, hydrogel, multiphase solution, vesicular dispersion or in any other known form of topical composition.

In certain embodiments, the topical composition may be formulated so that it can be applied, for example, via pen, tube, bottle, brush, stick, sponge, cotton swab, towelette (wipe), sprayer, dropper, hand or finger.

The composition may be formulated in a variety of product forms, such as, for example, a lotion, cream, serum, spray, aerosol, liquid cake, solid bar, ointment, essence, gel, paste, patch, pencil, powder, towelette, soap, shampoo, conditioner, stick, foam, mousse, elixir or concentrate. In preferred embodiments, the composition is formulated so that is particularly suitable for topical administration to the scalp.

### Methods of Promoting Hair Growth

In certain embodiments, the present invention provides methods for promoting hair growth, wherein a topical composition of the present invention is applied directly to an area of a subject's the skin where hair loss has occurred and/or where hair growth is desired, e.g., scalp, eyelids, face, limbs or torso.

In some embodiments, the methods can enhance scalp health, prevent hair loss, and/or stimulate hair growth for subjects in need thereof. In some embodiments, the subject is experiencing hair loss as a result of pattern baldness, chemotherapy, a skin injury, a burn, a scar or a medication side-effect.

In some embodiments, “applying” the composition can spreading, spraying or otherwise contacting the composition with the skin or hair. Reference to “skin” herein is meant to include the scalp in addition to other external parts of the body. In some embodiments, the composition is rubbed or massaged into the skin. In some embodiments, the composition is left on the skin to absorb completely, while in other embodiments, the composition is applied to the skin for a therapeutically-effective amount of time and then rinsed or removed from the skin using, for example, water or a cloth.

In certain embodiments, the topical composition is applied from zero to ten times daily, preferably at least once per day or at least once every other day. In some embodiments, the topical composition is applied daily or every other day for an indefinite period of time, e.g., for at least one, two, three weeks, or longer, in order to achieve and/or maintain a desired level of hair growth.

In one embodiment, the composition is applied in an amount from about 0.001 to about 100 mg per cm² of skin, more typically from about 0.01 to about 20 mg/cm², or from about 0.1 to about 10 mg/cm². More or less may be used, however, depending upon the size of the area of skin to be treated.

In certain embodiments, the method can further comprise the application of additional compositions for enhancing hair growth and/or for supplementing the activity of the topical composition as part of a hair growth regimen. For example, in one embodiment, the method can comprise cleansing the scalp or other area of the skin experiencing hair loss with a cleansing composition comprising a mixture of SLP and MEL.

The added cleansing step can be carried out, for example, from once daily to once weekly. In certain embodiments, cleansing is performed immediately prior to application of the topical composition. In certain embodiments, cleansing is performed several minutes to several hours prior to or after application of the topical composition, e.g., from 5 minutes to 12 hours before or after.

In certain embodiments, the cleansing composition comprises about 150 to 300 ppm, or about 200 ppm of SLP comprising a 70:30 ratio of linear to lactonic SLP. In certain embodiments, the cleansing composition comprises about 1 to 10 ppm, or about 5 ppm of MEL . . .

The cleansing composition can comprise additional components, including, for example, a carrier, moisturizers, fragrances, colorants, and others as are described above in relation to the topical composition for promoting hair growth. In certain embodiments, the cleansing composition is a shampoo, conditioner, or tonic for the hair.

In certain embodiments, the application of the topical composition and/or the cleanser can also be supplemented with administration of an oral compound, such as a DHT (dihydrotestosterone) blocker or 5-alpha-reductase inhibitor. DHT blockers can include, for example, alfatradiol, dutasteride, cpristeride and finasteride.

DHT is an adrogen derived from testosterone in the male body, and is needed for the development and maintenance of male sex characteristics, such as musculature and facial hair. Testosterone converts to DHT via 5-alpha-reductase, which is held in the oil glands of hair follicles. In some instances, subjects with pattern baldness experience shrinkage of hair follicles due to binding of the follicles by excess DHT; thus, in some embodiments, a DHT blocker can help prevent hair loss through this mechanism.

In some embodiments, the oral DHT blocker is administered to the subject daily at a dosage rate determined by a physician or pharmacist.

Advantageously, in some embodiments, the use of a multi-part regimen can lead to longer-lasting consistent results for subject experiencing hair loss.

### Growth of Microbes and Production of Microbial Growth By-Products

The subject invention provides methods for cultivating microorganisms and production of microbial metabolites and/or other by-products of microbial growth. As used herein “fermentation” refers to growth of cells under controlled conditions. The growth could be aerobic or anaerobic.

In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, and/or sodium carbonate. These inorganic salts may be used independently or in a combination of two or more.

In some embodiments, the method for cultivation may further comprise adding additional acids and/or antimicrobials in the liquid medium before and/or during the cultivation process. Antimicrobial agents or antibiotics are used for protecting the culture against contamination. Additionally, antifoaming agents may also be added to prevent the formation and/or accumulation of foam during cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control bacterial growth.

In one embodiment, the subject invention provides methods of producing a microbial metabolite, including, for example, a biosurfactant, enzyme and/or other protein, by cultivating a microbe strain of the subject invention under conditions appropriate for growth and production of the metabolite. The metabolite content of the resulting culture can be, for example, at least 20%, 30%, 40%, 50%, 60%, 70%, 80%, or 90%.

The biomass content of the fermentation broth may be, for example from 5 g/l to 180 g/l or more, or 10 g/l to 150 g/l.

The microbial growth by-product produced by microorganisms of interest may be retained in the microorganisms or secreted into the liquid medium. In another embodiment, the method for producing microbial growth by-product may further comprise steps of concentrating and purifying the microbial growth by-product of interest. In a further embodiment, the liquid medium may contain compounds that stabilize the activity of microbial growth by-product.

In one embodiment, all of the microbial culture is removed upon the completion of the cultivation (e.g., upon, for example, achieving a desired cell density, or density of a specified metabolite in the medium). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a cell-free broth or contain cells. In this manner, a quasi-continuous system is created.

Advantageously, the method does not require complicated equipment or high energy consumption. The microorganisms of interest can be cultivated at small or large scale on site and utilized, even being still-mixed with their media. Similarly, the microbial metabolites can also be produced at large quantities at the site of need.

The microorganisms can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In certain embodiments, the microbes are capable of producing amphiphilic molecules, enzymes, proteins and/or biopolymers. Microbial biosurfactants, in particular, are produced by a variety of microorganisms such as bacteria, fungi, and yeasts, including, for example, Agrobacterium spp. (e.g., A. radiobacter); Arthrobacter spp.; Aspergillus spp.; Aureobasidium spp. (e.g., A. pullulans); Azotobacter (e.g., A. vinelandii, A. chroococcum); Azospirillum spp. (e.g., A. brasiliensis); Bacillus spp. (e.g., B. subtilis, B. amyloliquefaciens, B. pumillus, B. coagulans, B. cereus, B. licheniformis, B. firmus, B. laterosporus, B. megaterium); Blakeslea; Candida spp. (e.g., C. albicans, C. rugosa, C. tropicalis, C. lipolytica, C. torulopsis); Clostridium (e.g., C. butyricum, C. tyrobutyricum, C. acetobutyricum, and C. beijerinckii); Campylobacter spp.; Cornybacterium spp.; Cryptococcus spp.; Debaryomyces spp. (e.g., D. hansenii); Entomophthora spp.; Flavobacterium spp.; Gordonia spp.; Hansenula spp.; Hanseniaspora spp. (e.g., H. uvarum); Issatchenkia spp; Kluyveromyces spp.; Meyerozyma spp. (e.g., M. guilliermondii); Mortierella spp.; Mycorrhiza spp.; Mycobacterium spp.; Nocardia spp.; Pichia spp. (e.g., P. anomala, P. guilliermondii, P. occidentalis, P. kudriavzevii); Phycomyces spp.; Phythium spp.; Pseudomonas spp. (e.g., P. aeruginosa, P. chlororaphis, P. putida, P. florescens, P. fragi, P. syringae); Pseudozyma spp. (e.g., P. aphidis); Ralslonia spp. (e.g., R. eulropha); Rhodococcus spp. (e.g., R. erythropolis); Rhodospirillum spp. (e.g., R. rubrum); Rhizobium spp.; Rhizopus spp.; Saccharomyces spp. (e.g., S. cerevisiae, S. boulardii sequela, S. torula); Sphingomonas spp. (e.g., S. paucimobilis); Starmerella spp. (e.g., S. bombicola); Thraustochytrium spp.; Torulopsis spp.; Ustilago spp. (e.g., U. maydis); Wickerhamomyces spp. (e.g., W. anomalus); Williopsis spp.; and/or Zygosaccharomyces spp. (e.g., Z. bailii).

In one embodiment, the method utilizes a yeast, such as, for example, Wickerhamomyces anomalus, Pseudozyma aphidis, Starmerella bombicola, Pichia kudriavzevii or Pichia guilliermondii (Meyerozyma guilliermondii). These yeasts are effective producers of various amphiphilic molecules, including glycolipids, enzymes and other useful metabolites. In a specific embodiment, the method utilizes W. anomalus.

Other microbial strains including, for example, other strains capable of accumulating significant amounts of, for example, amphiphilic molecules, can be used in accordance with the subject invention. Additional metabolites useful according to the present invention include mannoprotein, beta-glucan and other molecules that have bio-emulsifying and surface/interfacial tension-reducing properties.

### Preparation of Microbe-Based Products

One microbe-based product of the subject invention is simply the fermentation broth containing the microorganism and/or the microbial metabolites produced by the microorganism and/or any residual nutrients. The product of fermentation may be used directly without extraction or purification.

However, extraction and purification can be easily achieved using standard extraction and/or purification methods or techniques described in the literature. For example, in certain embodiments, the microbe-based product comprises simply the by-products of microbial growth, either in crude or purified form. In particular embodiments, the by-products are biosurfactants produced by the microorganisms grown according to the subject invention.

The microbes and/or broth resulting from the microbial growth can be removed from the growth vessel and transferred via, for example, piping for immediate use.

In other embodiments, the composition (microbes, broth, or microbes and broth) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

In certain embodiments, the compositions of the subject invention have advantages over, for example, biosurfactants alone, including one or more of the following: high concentrations of mannoprotein as a part of yeast cell wall's outer surface (mannoprotein is a highly effective bioemulsifier capable of reaching up to an 80% emulsification index); the presence of biopolymer beta-glucan (an emulsifier) in yeast cell walls; the presence of biosurfactants in the culture, which are capable of reducing both surface and interfacial tension; and the presence of metabolites (e.g., lactic acid, ethanol, etc.).

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Example of such additives include surfactants, emulsifying agents, lubricants, buffering agents, solubility controlling agents, pHI adjusting agents, preservatives, stabilizers and ultra-violet light resistant agents.

In one embodiment, the composition may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture thereof.

In one embodiment, additional components such as an aqueous preparation of a salt, such as sodium bicarbonate or carbonate, sodium sulfate, sodium phosphate, sodium biphosphate, can be included in the formulation.

Advantageously, in accordance with the subject invention, the microbe-based product may comprise the medium in which the microbes were grown. The product may be, for example, at least, by weight, 1%, 5%, 10%, 25%, 50%, 75%, or 100% growth medium. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

On the other hand, a biosurfactant composition can typically be stored at ambient temperatures.

### REFERENCES

Park, S.; Lee, J. “Modulation of Hair Growth Promoting Effect by Natural Products.” Pharmaceutics 2021, 13, 2163, https://doi.org/10.3390/pharmaceutics13122163
Woodward et al. “Composition of enhancing hair growth.” U.S. Pat. No. 9,700,503 B2.
“Composition for preventing hair loss or stimulating hair growth comprising sophorolipid as effective component.” KR Pat. No. 101931299 B1

1. A topical composition comprising one or more biosurfactants and a pharmacologic or naturopathic hair growth promoter, and, optionally, a dermatologically-acceptable carrier.

2. The topical composition of claim 1, wherein the one or more biosurfactants are glycolipids selected from sophorolipids (SLP) and mannosylerythritol lipids (MEL).

3. The topical composition of claim 2, comprising a mixture of SLP and MEL.

4. The topical composition of claim 2, wherein the SLP comprises a mixture of linear and lactonic SLP at a ratio of 70:30.

5. The topical composition of claim 2, comprising 100 to 250 ppm of SLP.

6. The topical composition of claim 2, comprising 1 to 10 ppm of MEL.

7. The topical composition of claim 1, wherein the pharmacologic hair growth promoter is minoxidil or bimatroprost.

8. The topical composition of claim 7, comprising 5% minoxidil.

9. The topical composition of claim 1, comprising 200 ppm SLP, 5 ppm MEL and 5% minoxidil, wherein the SLP comprise a mixture of linear and lactonic SLP at a ratio of 70:30.

10. (canceled)

11. The topical composition of claim 1, further comprising live or inactive cells of a microbe selected from the group consisting of Wickerhamomyces anomalus NRRL Y-68030, Starmerella bombicola, Meyerozyma guilliermondii, Pseudozyma aphidis, Bacillus coagulans, Bacillus amyloliquefaciens NRRL B-67928 and B. subtilis B4 NRRL B-68031.

12. The topical composition of claim 1, wherein the composition is formulated as a lotion, cream, gel, ointment, liquid, wipe, soap, shampoo, conditioner, tonic, mousse, foam, or spray, and wherein the formulation is suitable for direct application to skin, scalp and hair.

13. A method for promoting hair growth, which comprises applying a topical composition of claim 1, to an area of a subject's skin at which hair growth is desired.

14. The method of claim 13, wherein the SLP comprises a mixture of linear and lactonic SLP at a ratio of 70:30.

15. The method of claim 13, wherein the topical composition comprises 100 to 250 ppm of SLP.

16. The method of claim 13, wherein the topical composition comprises 1 to 10 ppm of MEL.

17. The method of claim 13, wherein the pharmacologic hair growth promoter is minoxidil or bimatroprost.

18. The method of claim 13, wherein the topical composition comprises 5% minoxidil.

19. The method of claim 13, which comprises applying 200 ppm SLP, 5 ppm MEL and 5% minoxidil to the subject's skin, wherein the SLP comprise a mixture of linear and lactonic SLP at a ratio of about 70:30.

20. The method of claim 13, wherein the subject is experiencing hair loss as a result of alopecia, chemotherapy, a skin injury, a burn or a medication side-effect.

21-29. (canceled)

30. The method of claim 13, further comprising administering alfatradiol, dutasteride, epristeride or finasteride.

31. (canceled)

32. A topical composition comprising at least one glycolipid selected from sophorolipids (SLP) and mannosylerythritol lipids (MEL) and a microbial culture supernatant, and a dermatologically-acceptable carrier.

33. (canceled)

34. The topical composition of claim 33, comprising a mixture of SLP and MEL.

35. The topical composition of claim 33, wherein the SLP comprises a mixture of linear and lactonic SLP at a ratio of 70:30.

36-37. (canceled)

38. The topical composition of claim 32, wherein the microbial culture supernatant is collected from cultivation of a B. coagulans.

39-42. (canceled)
